(19) United States
(12) Patent Application Publication
(10) Pub. No.: US 2022/0204551 A1
(43) Pub. Date: Jun. 30, 2022

(54) METHOD FOR THE PURIFICATION OF LIPOGLYCOPEPTIDE ANTIBIOTICS

(71) Applicant: HUVEPHARMA ITALIA S.R.L., Garessio (CN) (IT)

(72) Inventors: Carmine CAPOZZOLI, Gerenzano (Varese) (IT); Alberto TERRANEO, Gerenzano (Varese) (IT)

(21) Appl. No.: 17/607,639

(22) PCT Filed: Apr. 24, 2020
(86) PCT No.: PCT/IB2020/053884

§ 371 Date: Oct. 29, 2021

(30) Foreign Application Priority Data
Apr. 30, 2019 (IT) 102019000006442

(51) Int. Cl. C07K 1/16; C07K 9/00

(52) U.S. Cl. CPC C07K 1/16; C07K 9/008

(57) ABSTRACT

Method for purifying at least one lipoglycopeptide antibiotic comprising the steps of: i) dissolving said at least one lipoglycopeptide antibiotic in an aqueous solution to form a mixture, ii) loading said mixture into a chromatographic column comprising a stationary phase, wherein said stationary phase comprises silica functionalized with organic pendants, iii) eluting the mixture loaded in step ii) using an eluent composition comprising a water-soluble organic solvent obtaining eluate fractions, iv) selecting the eluate fractions containing the at least one purified lipoglycopeptide antibiotic.

### FIELD OF THE INVENTION

The present description relates to methods for purifying active principles. More specifically, the description relates to methods for purifying lipoglycopeptide antibiotics.

### BACKGROUND

Dalbavancin is included among the lipoglycopeptide antibiotics known today. Dalbavancin is a second generation semisynthetic lipoglycopeptide with activity towards a broad spectrum of Gram-positive pathogenic microorganisms. The use of dalbavancin for treating Gram-positive bacterial infections is, for example, described in documents U.S. Pat. No. 6,900,175 B2, US 2005/0004050 A1 and US 2009/0298749 A1.

The semi-synthesis of the dalbavancin molecule starting from the precursor of natural origin A40926—obtained by fermentation carried out by the microorganism Nonomuraea gerenzanensis—is described, for example, in the document U.S. Pat. No. 6,900,175 B2.

Methods known to date for purifying the unrefined (crude) compound A-40926 and dalbavancin are carried out by adsorption chromatography on polyamide. These methods, however, can present critical issues due to sub-optimal operating conditions, and the obtaining of a final product that may contain impurities. In particular, the choice of using adsorption chromatography on polyamide may require a complex fractionation work with a step gradient using carbonate/bicarbonate buffered solutions. Furthermore, these operating conditions can lead to the formation of an isomerization impurity that is difficult to control. There is, therefore, the need to provide complex and repeated fractionation steps which may have the disadvantage of reducing the yield of the method in relation to obtaining a product whose purity can be increased.

### SUMMARY OF THE INVENTION

The present description is intended to provide purifying methods for lipoglycopeptide antibiotics that are simple, with reduced environmental impact and capable of providing high purity products.

According to the present description, the above object is achieved thanks to the subject to which specific reference is made in the following claims, intended as an integral part of the present description.

One embodiment of the present description provides a method for purifying at least one lipoglycopeptide antibiotic comprising the steps of:

i) dissolving said at least one lipoglycopeptide antibiotic in an aqueous solution to form a mixture,

ii) loading said mixture into a chromatographic column comprising a stationary phase, wherein said stationary phase comprises silica functionalized with organic pendants,

iii) eluting the mixture loaded in step ii) using an eluent composition comprising a water-soluble organic solvent obtaining eluate fractions,

iv) selecting the eluate fractions containing the at least one purified lipoglycopeptide antibiotic.

In one or more embodiments, the silica functionalized with organic pendants may comprise octadecyl silyl derivatized silica, octyl silyl derivatized silica, exylphenyl silyl derivatized silica, or butyl silyl derivatized silica.

The silica functionalized with organic pendants of the stationary phase may have a particle size of less than 50 μm, preferably less than 20 μm.

The stationary phase may also be conditioned prior to the loading step ii) with a mobile phase that comprises at least one compound selected in the group consisting of ammonium formate, ammonium acetate or triethylamine salts with formic acid, acetic acid, trifluoroacetic acid. This mobile phase may have a pH between 5.0 and 7.0.

The method may, furthermore, comprise the steps of:

v) concentrating the eluate fractions collected in step iv) to obtain a concentrate,

vi) optionally dialyzing the concentrate obtained in step v),

vii) precipitating said concentrate, optionally dialyzed in step vi), with a precipitation solution comprising at least one organic solvent to obtain a precipitate,

viii) drying the precipitate obtained in step vii).

### BRIEF DESCRIPTION OF THE DRAWINGS

The invention will now be described, by way of example, with reference to the attached figures, wherein:

FIG. 1 illustrates the chromatographic profile of the purification of A40926 conducted according to embodiments of the present description,

FIG. 2 illustrates the chromatographic profile of the purification of dalbavancin conducted according to embodiments of the present description,

FIG. 3 illustrates the chromatographic profile of the purification of dalbavancin conducted according to embodiments of the present description,

FIG. 4 illustrates the chromatographic profile of the purification of dalbavancin conducted according to embodiments of the present description,

FIG. 5 illustrates the analytical HPLC profile of dalbavancin currently commercially available,

FIG. 6 illustrates the analytical HPLC profile of purified dalbavancin according to embodiments of the present description.

### DETAILED DESCRIPTION OF PREFERRED EMBODIMENTS

In the following description, numerous specific details are provided to allow a thorough understanding of embodiments. The embodiments can be implemented without one or more of the specific details or with other methods, components, materials etc. In other cases, well-known structures, materials or operations are not shown or described in detail to avoid confusing aspects of the embodiments.

Reference throughout the present disclosure to “one embodiment” or “an embodiment” indicates that a particular aspect, structure or characteristic described with reference to the embodiment is included in at least one embodiment. Thus, forms of the expressions “in one embodiment” or “in an embodiment” at various points throughout the present description do not necessarily all refer to the same embodiment. Moreover, the particular aspects, structures or characteristics can be combined in any convenient way in one or more embodiments. The titles provided in this description are for convenience only and do not interpret the scope or object of the embodiments.

The Inventors of this application have identified specific operating conditions that favor the obtainment of effective purifications of lipoglycopeptide antibiotics by means of a simple method, characterized by lower costs and reduced environmental impact compared to the lipoglycopopeptide antibiotic purification methods known in the art.

One embodiment of the present description provides a method for purifying at least one lipoglycopeptide antibiotic comprising the steps of:

i) dissolving said at least one lipoglycopeptide antibiotic in an aqueous solution to form a mixture,

ii) loading said mixture into a chromatographic column comprising a stationary phase, wherein said stationary phase comprises silica functionalized with organic pendants,

iii) eluting the mixture loaded in said step ii) using an eluent composition comprising a water-soluble organic solvent obtaining eluate fractions,

iv) selecting the eluate fractions containing the at least one purified lipoglycopeptide antibiotic.

In one or more embodiments, the selected eluate fractions have a purity equal to or greater than 90%.

Lipoglycopeptide antibiotics that can be purified using the method described in the present description may, for example, be selected in the group consisting of A40926, dalbavancin, teicoplanin, mideplanin (MDL-62873), and ramoplanin.

Dalbavancin and the compound A40926 (its intermediate) to be subjected to the method described in the present description can be obtained by methods known in the art. A40926 is a fermentation product which can be isolated, for example, from a fermentation broth of Nonomuraea gerenzanensis ATCC 39727 by filtration, affinity chromatography and concentration by azeotropic distillation with butanol, followed by the addition of petroleum ether to precipitate the unrefined product as, for example, described in the document U.S. Pat. No. 4,935,238.

Dalbavancin can be obtained, for example, by amidation of the monomethyl ester of A40926 and subsequent hydrolysis, as described in the document US 2004/0142883 A1.

According to the purification method described here, the antibiotic to be subjected to purification is dissolved in an aqueous solution to obtain a mixture.

In one or more embodiments, said mixture may comprise the at least one antibiotic to be subjected to purification in a concentration of between 25 g/l and 45 g/l.

In one embodiment, step i) may comprise adding a component selected in the group consisting of formic acid, sulfuric acid, acetic acid, trifluoroacetic acid, hydrochloric acid, and phosphoric acid, preferably hydrochloric acid, to the aqueous solution. For example, the method may comprise the step of adding an acid component selected in the group consisting of formic acid, sulfuric acid, acetic acid, trifluoroacetic acid, hydrochloric acid, and phosphoric acid to the aqueous solution, when the antibiotic to be purified is dalbavancin, or teicoplanin, or mideplanin (MDL-62873), or ramoplanin.

In another embodiment, step i) may comprise adding a component selected in the group consisting of ammonia, sodium hydroxide, potassium hydroxide, sodium carbonate, potassium carbonate, sodium bicarbonate, and potassium bicarbonate, preferably sodium hydroxide. For example, the method may comprise the step of adding a basic component to the aqueous solution selected in the group consisting of ammonia, sodium hydroxide, potassium hydroxide, sodium carbonate, potassium carbonate, sodium bicarbonate, and potassium bicarbonate, preferably sodium hydroxide when the antibiotic to be purified is A40926.

The mixture thus obtained is loaded onto a chromatographic column with a stationary phase which may comprise silica functionalized with organic pendants.

The expression silica functionalized with organic pendants means silica to which aliphatic or aryl aliphatic organic chains have been covalently linked, through siloxane bonds.

The silica functionalized with organic pendants may be selected in the group consisting of octadecyl silyl derivatized silica, octyl silyl derivatized silica, exylphenyl silyl derivatized silica, and butyl silyl derivatized silica.

Advantageously, the ratio between the quantity of the loaded mixture and the volume of the stationary phase is between 0.1 g/l and 1.6 g/l.

The inventors of the present application have observed that particularly advantageous results are obtained when the stationary phase comprises octadecyl silyl derivatized silica (C18), preferably with a particle size lower than 50 μm, more preferably lower than 20 μm.

The stationary phase can be conditioned, prior to the loading step ii), with a mobile phase which may have a pH between 5.0 and 7.0.

For conditioning the stationary phase, this mobile phase may comprise an aqueous solution of at least one compound selected in the group consisting of ammonium formate, ammonium acetate or an aqueous solution comprising triethylamine salts with formic acid, acetic acid, and trifluoroacetic acid.

In one or more embodiments, the at least one compound of the mobile phase has a concentration of between 0.01% and 10% w/v of the aqueous solution.

In one or more embodiments, the mobile phase comprises an aqueous solution of ammonium formate, preferably in a molar concentration of between 0.0025M and 0.05M.

In one or more embodiments, the mobile phase may comprise an aqueous solution of triethylamine and formic acid, preferably each in a concentration comprised between 0.005% w/w and 0.2% w/w.

After loading the mixture comprising the antibiotic to be subjected to purification, the elution step follows with an eluent composition, which may comprise at least one water-soluble organic solvent selected in the group consisting of methanol, propanol, isopropanol, acetonitrile, and acetone. Preferably, the eluent composition comprises acetonitrile.

The concentration of the water-soluble organic solvent can be between 10% and 90% v/v of the eluent composition.

In one or more embodiments, the eluent composition may comprise at least one additional compound selected in the group consisting of triethylamine, formic acid, acetic acid, trifluoroacetic acid, heptafluorobutyric acid, methanesulfonic acid, ammonium formate, and ammonium acetate. This additional component can be present in a concentration comprised between 0.005% w/w and 0.2% w/w.

The elution step iii) can be carried out at a pH between 5.0 and 7.0, preferably between 5.2 and 6.5.

The elution step may be carried out at a flow of between 3 and 5 bed volumes/hour.

In one or more embodiments, the eluent composition may comprise said water-soluble organic solvent, preferably acetonitrile, in a concentration increasing over time, at least in one interval of conduction time of the elution step iii) or along the entire elution step iii). The concentration, for example, of acetonitrile, may increase from a minimum value of 10% to a maximum value of 90% v/v of the eluent composition in said at least one interval of conduction time of the elution step iii) or along the entire elution step iii). The eluent composition comprising said at least one organic solvent in increasing concentration may be obtained by mixing i) a pure solution of said at least one organic solvent, for example, acetonitrile, with ii) an aqueous solution. The mixing can be carried out using a pure solution quantitative ratio of said at least one organic solvent:aqueous solution variable over the conduction time of the eluent step. This quantitative ratio may vary, for example, between a minimum value of 0.1 (pure solution:aqueous solution ratio 10:90) to a maximum value of 9 (pure solution:diluent solution 90:10). The aqueous solution may comprise at least one compound selected in the group consisting of triethylamine, formic acid, acetic acid, trifluoroacetic acid, heptafluorobutyric acid, methanesulfonic acid, ammonium formate, and ammonium acetate, preferably in a concentration of between 0.005% w/w and 0.2% w/w.

The method may comprise a step of selecting the eluate fractions and collecting the selected eluate fractions. Selecting eluate fractions is based on the degree of purity of the antibiotic subjected to the method in question. Preferably, the method envisages selecting eluate fractions with a purity greater than or equal to 90% (greater than or equal to 90% considering the sum of the compounds of interest contained in the mixture).

The selection of the eluate fractions to be concentrated is carried out by HPLC analysis using, for example, the method reported in the document U.S. Pat. No. 6,900,175 B2, or official pharmacopoeial compendial methods.

The selected and collected eluate fractions can consequently be subjected to a concentration step to obtain a concentrate.

This concentration step of the fractions may be advantageously carried out using, for example, a membrane nanofiltration system with a molecular cut-off ranging from 100 Da and 1500 Da.

The concentration step of the fractions allows a quantity of purified antibiotic to be obtained in the concentrate ranging from 10 mg/ml and 150 mg/ml, preferably from 20 mg/ml to 80 mg/ml.

The method may also comprise a step of adjusting the pH of the concentrate to a value from 2.5 to 4.0 by adding, in the concentrate, an aqueous solution of an acid, for example, hydrochloric acid. In one or more embodiments, the aqueous solution of an acid may comprise 15% w/w of hydrochloric acid.

The excess water in the concentrate can be eliminated, for example, by means of nanofiltration or reverse osmosis.

In one or more embodiments, the method may include a dialyzing step of the concentrate. This step allows elimination from the concentrate of the at least one compound contained in the mobile phase used for conditioning. Furthermore, this dialyzation step may allow removal of traces of the at least one organic solvent and/or of the possible additional compound selected in the group consisting of triethylamine, formic acid, acetic acid, trifluoroacetic acid, heptafluorobutyric acid, methanesulfonic acid, ammonium formate, and ammonium acetate used in the eluent composition of the elution step.

In one or more embodiments, the method may also comprise at least one step of precipitating the concentrate with a precipitation solution comprising at least one organic solvent selected in the group consisting of acetonitrile, acetone, isopropanol, propanol and ethanol, preferably acetone. The concentrate can be resuspended in water and then in acetone to obtain the complete precipitation of the required product.

In the precipitation step, the pH can be adjusted to a value between 4.0 and 6.0, preferably 5, with an alkaline aqueous solution, for example, of sodium hydroxide. For example, the alkaline solution may comprise 20% w/w sodium hydroxide.

The precipitate obtained can be filtered and washed with an organic solvent, for example acetone.

A step follows in which the precipitate is dried, for example, under vacuum at a temperature of 30° C.

In one or more embodiments, the precipitate may be freeze-dried, for example, at a temperature of 15° C. and a pressure of 50 microbar.

The described method, compared to methods known in the art, has a number of advantages. For example, the choice of the specific stationary phase and of an eluent comprising a water soluble organic solvent allows a more effective purification to be obtained.

The method, in fact, allows elimination of colored compounds, less polar impurities of the antibiotic of interest and—at the same time—more polar ones, which would otherwise require separate purification steps.

In addition, the concentration step of the selected eluate fractions is simple and fast thanks to the use, for example, of nanofiltration; the choice of this operating condition in combination with controlling the working pH allows avoiding the degradation of the products obtained, degradation that can occur, for example, when the purification method involves concentration steps obtained by hot distillation.

The described method allows a high yield to be obtained which is between 80.0% and 96.0%.

In addition, as will be evident below, the purity of the product obtained is significantly higher than the purity of products available on the market and obtained with the methods known in the art.

### EXAMPLES

### Example 1—Purification Method of the Compound A40926

On a preparative HPLC chromatographic column of 10 cm diameter and 60 cm packed height with stationary phase compound of octadecyl silyl derivatized silica (C18) with a particle size of 15 μm (Luna C18, Phenomenex), conditioned with a mobile phase comprising 0.025 M aqueous pH 6.5 ammonium formate (CarloErba) (mobile phase), 200 ml of an aqueous solution of 20 g of unrefined A40926 (4.9 g antibiotic activity) was loaded.

Unrefined A-40926 was obtained by submerged fermentation of N. gerenzanensis, as described in the document U.S. Pat. No. 4,935,238, followed by microfiltration of the harvest broth, concentration by nanofiltration and precipitation of the unrefined product, as described in the document U.S. Pat. No. 6,900,175 B2. In particular, the method for obtaining unrefined A-40926 comprises the steps described below. A cryotube of the N. gerenzanensis strain producing A40926 is used to inoculate a 500 mL (Erlenmeyer) flask containing 50 mL of culture medium A composed as follows: 1 g/l Dextrose (Roquette); 24 g/l Soluble starch (Difco-BD); 5 g/l Yeast extract (Constantine); 5 g/l Tryptose (Difco-BD); 4 g/l calcium carbonate (Imerys). The culture is incubated at 28° C. on a 240 rpm rotary shaker.

After 72 h, 10% of the culture is transferred to a 500 mL (Erlenmeyer) flask containing 100 ml of culture medium B with the following composition: 25 g/l Dextrose (Roquette); 4 g/l Yeast autolysate (Constantine); 20 g/l Soybean meal (Mucedola); 1.25 g 1 sodium chloride (Carlo Erba); 5 g/l calcium carbonate (Imerys); 0.6 g/l Defoamer (Momentive Performance Materials Inc); (pH 7.6 before sterilization).

After 96 hours, 3% of the culture is transferred to a 20 l prefermenter containing 16 l of culture medium B. The fermenter is stirred at about 900 rpm with a sterile air flow equal to 4 liters per minute. After 72 hours, the content is transferred to a 200 l fermenter containing 145 l of culture medium B. After 168 hours, the maximum production of A40926 and its acetylated derivative is reached (about 1 g/l of A40926).

The culture broth is treated with 20% sodium hydroxide up to pH 11.4, keeping the temperature at 23° C. It is kept at this temperature for 6 hours, then it is cooled to 15° C. and it is subjected to microfiltration on ceramic membranes (Koch, 0.1 micron). The permeate, containing A40926, is brought to pH 8.5 with 15% hydrochloric acid and subjected to concentration by means of nanofiltration (membranes 250 Da, Koch) until a solution with a concentration of 40 g/l of A40926 is obtained. The solution is then treated with 9 volumes of acetone obtaining the precipitation of unrefined A40926, which is then dried under vacuum to a water content of less than 10%.

The column was then eluted at 320 ml/min with an eluent composition comprising acetonitrile (CarloErba) in an increasing concentration over the elution conduction time. This eluent composition was obtained following the mixing in time-varying quantitative ratios, as illustrated in Table 1, of a first solution (mobile phase A) comprising demineralized water with the addition of 0.025M ammonium formate (CarloErba) at pH 6.5, and a second solution (mobile phase B) of pure acetonitrile (CarloErba).

TABLE 1

| Time | Mobile phase A | Mobile phase B |
|---|---|---|
| (min) | % (v/v) | % (v/v) |
| 0 | 80 | 20 |
| 5 | 75 | 25 |
| 45 | 69 | 31 |
| 54 | 69 | 31 |
| 55 | 10 | 90 |
| 70 | 10 | 90 |

The eluate was collected in 4 fractions as indicated in Table 2.

TABLE 2

| | Collection | | Fraction |
|---|---|---|---|
| Fraction | (min) | Concentration | weight |
| F1 | 32-36.5 | 0.049 g/l | 2615 g |
| F2 | 36.5-38.6 | 0.121 g/l | 2000 g |
| F3 | 38.6-46.3 | 1.864 g/l | 3530 g |
| F4 | 46.3-49.7 | 0.462 g/l | 2305 g |

The yield of the purification method was 93%.

The fractions containing A40926 that resulted in the specification were concentrated and dialyzed against demineralized water (10 diavolumes) with a nanofiltration system with a molecular cut-off of 250 Da (HydroAirResearch) until a concentration of A40926 in the concentrate of 20 g/l was obtained. Dialysis allows removal of the ammonium formate present in the eluent mixture.

The selection of the eluate fractions to be concentrated is carried out by HPLC analysis by choosing the fractions with purity greater than or equal to 90%, calculated on the sum of the areas of the peaks of the antibiotic subjected to purification.

The concentrate was brought to pH 3.8+/−0.2 with 15% w/w aqueous hydrochloric acid (CarloErba) and centrifuged to remove the water. The solid residue was resuspended with 20 volumes of water with respect to A40926 followed by 20 volumes of acetone (CarloErba), and the pH raised to 5 with 20% aqueous sodium hydroxide (CarloErba). Another 120 volumes of acetone (CarloErba) were then added, obtaining the complete precipitation of the required product. The solid was recovered by filtration, washed with 20 volumes of acetone (CarloErba) and dried under vacuum at 30° C. The yield of the process was 92.4%.

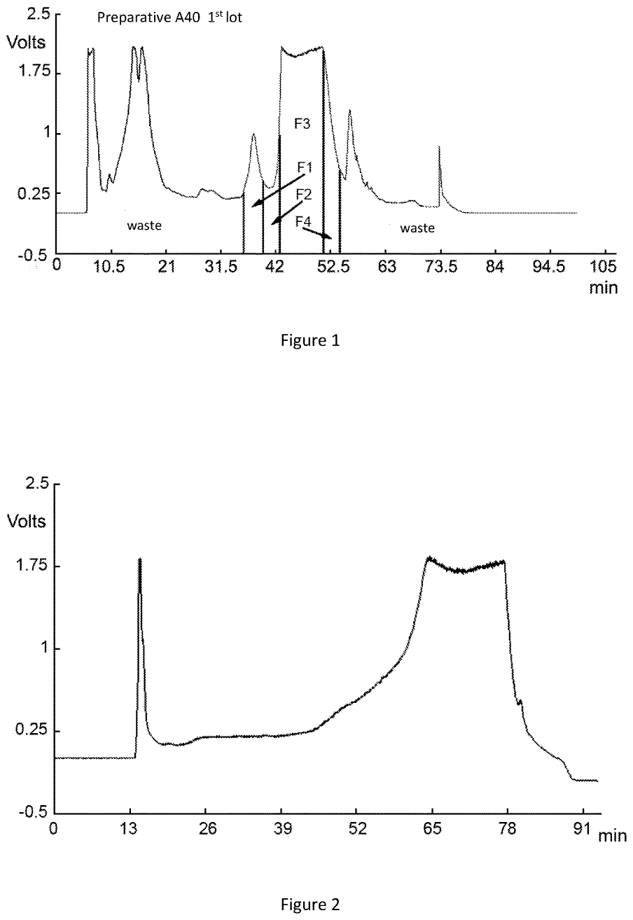

FIG. 1 illustrates the chromatographic profile (tracing of the UV signal read at 280 nm) of the purification of A40926 obtained by preparative HPLC carried out by applying the conditions of example 1. The vertical lines indicate the boundary between one collection fraction and the next. As can be seen immediately, the fractions F1 and F3 correspond to two main peaks of the chromatogram: they contain the active components of the antibiotic A40926. The impurities are eliminated in the fractions called “waste” and F2 and F4.

### Example 2—Purification Method of Dalbavancin

On a preparative HPLC chromatographic column of 10 cm diameter and 60 cm packed height with stationary phase compound of octadecyl silyl derivatized silica (C18) with a particle size of 15 μm (Phenomenex), conditioned with a mobile phase of 0.025 M aqueous pH 6.5 ammonium formate (CarloErba) (mobile phase), 680 ml of an aqueous solution of unrefined dalbavancin (7.3 g) was loaded.

Dalbavancin was obtained from A40926 through a sequence of reactions that provide protection such as monomethyl ester, amidation with 3-N,N-dimethylaminopropylamine final deprotection to give Dalbavancin as described in document U.S. Pat. No. 6,900,175 B2.

In particular, the method of obtaining dalbavancin comprises the steps of: methylation of A40926 in methanol (CarloErba) catalyzed by concentrated sulfuric acid (CarloErba) followed by precipitation of the methyl ester of A40926 by dilution with water and pH correction between 4.5 and 6.5 with triethylamine (CarloErba). Amidation of methyl ester into dimethyl sulfoxide (CarloErba) and methanol (CarloErba) with 3-N, N-dimethylaminopropylamine (SigmaAldrich) using dicyclohexylcarbodiimide (SigmaAldrich) as the condensing agent. The methyl ester amide obtained is hydrolyzed with 20% aqueous sodium hydroxide (CarloErba) to give an unrefined dalbavancin solution which, after correcting the pH to 3.5 with 15% aqueous hydrochloric acid (CarloErba), is used for the purification tests as such, or by preceding a dialysis against water to remove the organic solvents present (dimethyl sulfoxide and methanol).

The column was then eluted at 320 ml/min with an eluent composition comprising acetonitrile (CarloErba) in an increasing concentration over the elution conduction time. This eluent composition was obtained following the mixing, in time-varying quantitative ratios, as illustrated in Table 3, of a first solution (mobile phase A) comprising demineralized water with the addition of 0.025M aqueous ammonium formate (CarloErba) at pH 6.5, and a second solution (mobile phase B) of pure acetonitrile (CarloErba).

TABLE 3

| Time | Mobile phase A | Mobile phase B |
|---|---|---|
| (min) | % (v/v) | % (v/v) |
| 0 | 80 | 20 |
| 5 | 80 | 20 |
| 40 | 65 | 35 |
| 50 | 60 | 40 |
| 60 | 55 | 45 |
| 70 | 55 | 45 |
| 71 | 10 | 90 |
| 85 | 10 | 90 |

The eluate was collected in 7 fractions as described in Table 4.

TABLE 4

| | Collection | Concentration | Fraction volume |
|---|---|---|---|
| Fraction | (min) | (g/l) | (l) |
| F1 | 20-37 | — | 5.4 |
| F2 | 37-49.6 | 0.1 | 4.0 |
| F3 | 49.6-56 | 0.1 | 2.0 |
| F4 | 56-69 | 0.4 | 4.2 |
| F5 | 69-78 | 0.8 | 2.9 |
| F6 | 78-85 | 1.0 | 2.2 |
| F7 | 85-93 | 0.3 | 2.6 |

The yield of the purification method was found to be 95%.

The fractions containing dalbavancin, resulting in specification, were concentrated and dialyzed against demineralized water (10 diavolumes), with a nanofiltration system with a molecular cut-off of 250 Da (HydroAirResearch) until a concentration of dalbavancin in the concentrate of 80 g/l was obtained. The selection of the eluate fractions to be concentrated is carried out by HPLC analysis by choosing the fractions with purity greater than or equal to 90% calculated on the sum of the areas of the peaks of the compounds of interest.

The concentrate was then brought to pH 2.6+/−0.1 with 15% w/w aqueous hydrochloric acid (Carlo Erba). A total of 9 volumes of acetone (CarloErba) were then added to the dalbavancin solution in water at a temperature below 10° C. The solid is recovered after overnight incubation at 4° C. by filtration on a porous septum, washed with 1.5 volumes of acetone (CarloErba) and dried under vacuum (50 μbar) at 15° C. The yield of the method was 84.0%.

FIG. 2 illustrates the chromatographic profile (tracing of the UV signal read at 280 nm) of the purification of dalbavancin obtained by preparative HPLC carried out by applying the conditions of example 2.

The apparent absence of resolution is due to the extreme concentrations of the material that saturates the detector of the instrument. However, it is possible to note that in the initial and final parts of the chromatogram there are peaks, which testify successful separation of the impurities.

### Example 3—Purification of Dalbavancin

On a preparative HPLC chromatographic column of 10 cm diameter and 60 cm packed height with stationary phase compound of octadecyl silyl derivatized silica (C18) with a particle size of 15 μm (Phenomenex), conditioned with an aqueous solution of 0.1% v/v triethylamine (CarloErba) and 0.1% v/v formic acid (CarloErba) (mobile phase A), 680 ml of an aqueous solution of unrefined dalbavancin (7.3 g active) was loaded, obtained as described in Example 2.

The column was then eluted at 320 ml/min with an eluent composition comprising acetonitrile (CarloErba) in an increasing concentration over the elution conduction time. This eluent composition was obtained following the mixing, in quantitative ratios varying over time, as illustrated in Table 5, of a first solution (mobile phase A) comprising demineralized water with the addition of 0.1% v/v triethylamine (CarloErba) and 0.1% v/v formic acid (CarloErba) and a second solution (mobile phase B) of pure acetonitrile (CarloErba) supplemented with 0.1% v/v triethylamine (CarloErba) and 0.1% v/v formic acid (CarloErba).

TABLE 5

| Time | Mobile phase A | Mobile phase B |
|---|---|---|
| (min) | % (v/v) | % (v/v) |
| 0 | 80 | 20 |
| 5 | 75 | 25 |
| 40 | 70 | 30 |
| 50 | 60 | 40 |
| 60 | 60 | 40 |
| 65 | 10 | 90 |
| 76 | 10 | 90 |

The eluate was collected in 7 fractions as indicated in Table 6.

TABLE 6

| | Collection | Concentration | Fraction volume |
|---|---|---|---|
| Fraction | (min) | (g/l) | (l) |
| F1 | 28-32.5 | 0 | 1.42 |
| F2 | 32.5-37.5 | 0 | 1.57 |
| F3 | 37.5-40.5 | 0.1 | 0.94 |
| F4 | 40.5-43 | 0.3 | 0.82 |
| F5 | 43-47.2 | 0.1 | 1.32 |
| F6 | 47.2-51.5 | 4.1 | 1.32 |
| F7 | 51.5-57.5 | 0.8 | 1.87 |

The yield of the purification method was found to be 95%.

The fractions containing dalbavancin, resulting in specification, were concentrated and dialyzed against demineralized water (10 diavolumes), with a nanofiltration system with a molecular cut-off of 250 Da (HydroAirResearch) until a concentration of dalbavancin in the concentrate of 80 g/l was obtained. The concentrate was then brought to pH 2.6+/−0.1 with 15% w/w aqueous hydrochloric acid (CarloErba). The selection of the eluate fractions to be concentrated is carried out by HPLC analysis by choosing the fractions with purity greater than or equal to 90% calculated on the sum of the areas of the peaks of the compounds that make up the antibiotic.

A total of 9 volumes of acetone (CarloErba) were then added to the dalbavancin solution in water at a temperature below 10° C. The solid was recovered after overnight incubation at 4° C. by filtration on a porous septum, washed with 1.5 volumes of acetone (CarloErba) and dried under vacuum (50 μbar) at 15° C. The yield of the method was 84.0%.

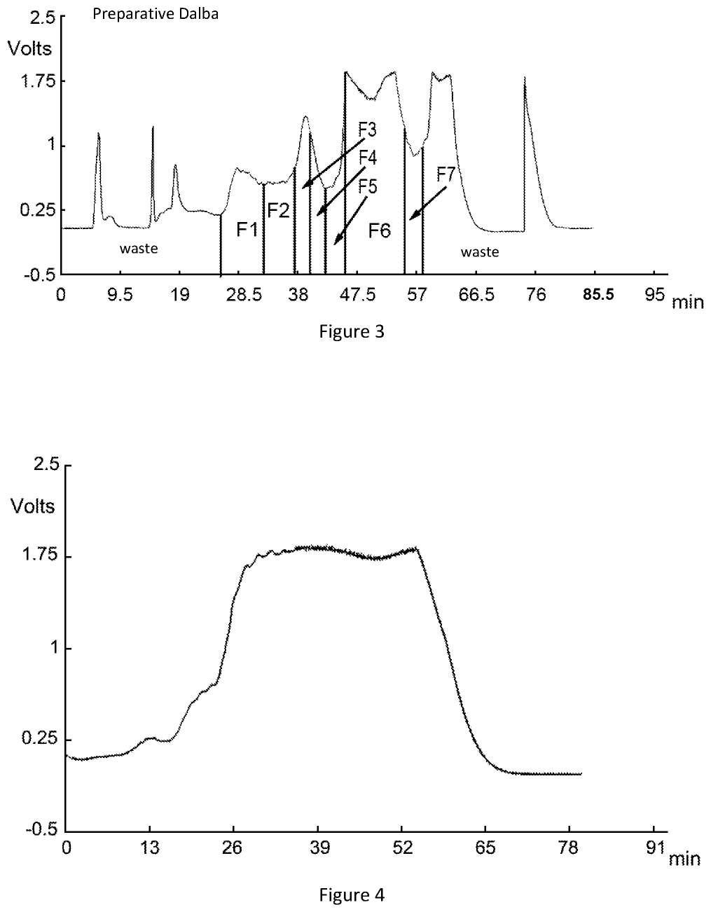

FIG. 3 illustrates the chromatographic profile (tracing of the UV signal read at 280 nm) of the purification of dalbavancin obtained by preparative HPLC carried out by applying the conditions of example 2. The vertical lines indicate the boundary between one collection fraction and the next.

The chromatographic profile shows the high resolution between the various components of the mixture which, therefore, allows the almost complete removal of the impurities present.

### Example 4—Purification of Dalbavancin

On a preparative HPLC chromatographic column of 10 cm diameter and 60 cm packed height with stationary phase compound of octadecyl silyl derivatized silica (C18) with a particle size of 15 μm (Phenomenex), conditioned with 0.025 M aqueous ammonium formate (CarloErba) pH 5.2 (mobile phase A), 680 ml of an aqueous solution of unrefined dalbavancin (active 7.3 g) was loaded, obtained as described in Example 2.

The column was then eluted at 320 ml/min with an eluent composition comprising acetonitrile (CarloErba) in an increasing concentration over the elution conduction time. This eluent composition was obtained following the mixing in time-varying quantitative ratios, as illustrated in Table 7, of a first solution (mobile phase A) comprising demineralized water with the addition of 0.025M aqueous ammonium formate (CarloErba) at pH 5.2 and a second solution (mobile phase B) of pure acetonitrile (CarloErba).

TABLE 7

| Time | Mobile phase A | Mobile phase B |
|---|---|---|
| (min) | (% v/v) | % (v/v) |
| 0 | 80 | 20 |
| 60 | 65 | 35 |
| 90 | 60 | 40 |
| 110 | 50 | 50 |
| 120 | 10 | 90 |
| 140 | 10 | 90 |

The eluate was collected in 13 fractions as described in Table 8.

TABLE 8

| | Collection | Concentration | Fraction volume |
|---|---|---|---|
| Fraction | (min) | (g/l) | (l) |
| F1 | 14.7-18 | 0 | 1.1 |
| F2 | 43-51 | 0 | 2.6 |
| F3 | 51-60 | 0 | 2.9 |
| F4 | 60-69 | 0.1 | 2.9 |
| F5 | 69-77 | 0.2 | 2.6 |
| F6 | 77-83 | 0.1 | 1.9 |
| F7 | 83-88 | 0.2 | 1.6 |
| F8 | 88-95 | 0.4 | 2.2 |
| F9 | 95-103 | 0.6 | 2.6 |
| F10 | 103-112 | 0.8 | 2.9 |
| F11 | 112-120 | 0.4 | 2.6 |
| F12 | 120-127 | 0.2 | 2.2 |
| F13 | 127-140 | 0 | 4.2 |

The yield of the purification method was found to be 95%.

FIG. 4 illustrates the chromatographic profile (tracing of the UV signal read at 280 nm) of the purification of Dalbavancin obtained by preparative HPLC carried out by applying the conditions of example 4. The apparent absence of resolution is due to the high concentration of the mixture subjected to purification. However, it is possible to note that impurities accumulate at the beginning and end of the peak, thus allowing recovery of the pure product in the central part of the peak.

Compared with purification methods known in the art, the method described in this application allows a greater overall yield to be obtained. For example, the purification method carried out by polyamide stationary phase adsorption chromatography (as described, for example, in US 2004/0142883 A1) has a weight/weight yield of API dalbavancin between 25% and 33% weight/weight starting from the intermediate A40926 present in the initial fermentation broth. The method described herein has a yield by weight/weight of API dalbavancin, starting from A40926 initially present in the fermentation broth, greater than 35% weight/weight, with a yield increase that varies between 5% and 60%, on average 32%.

Furthermore, the purity of the product obtained with the described method is significantly higher than that of the product obtained by purification with adsorption chromatography on polyamide, for example, described in document US 2004/0142883 A1.

Table 9 below reports the results obtained by analyzing, by means of HPLC, a commercial sample of Dalbavancin (Xydalba, Durata Therapeutics) and a sample obtained by applying the purification method described in this description. It is possible to note that the total correlated substances are reduced by 50% and, above all, the impurity mannosyl aglycone (MAG), which originates by decomposition of the Dalbavancin, without antibiotic activity, is reduced by 88% by simultaneously increasing the active component.

TABLE 9

| | Method | Method |
|---|---|---|
| | adsorption on | present |
| | polyamide | description |
| Parameter | [% area] | [% area] |
| Complex distribution of | | |
| the components | | |
| A₀ + A₁ | 3.2 | 1.2 |
| B₀ | 83.1 | 90.7 |
| B₁ + B₂ | 10.3 | 6.0 |
| Correlated substances | 0.2 | <0.2 |
| D₀ + D₁ | <0.2 | <0.2 |
| C₀ + C₁ | 1.7 | 0.2 |
| MAG | 0.6 | 0.5 |
| IsoB₀ | 0.5 | 0.2 |
| Trichloro | <0.2 | 0.3 |
| DesAA-1 | 0.3 | 0.5 |
| RRT 0.93 | <0.2 | <0.2 |
| RRT 1.3 | <0.2 | 0.3 |
| MA-A-1/RRT1.43 | <0.2 | 0.2(rrt1.16) |
| Single substance not specified | <0.2 | |
| Total of unspecified substances | 3.3 | 0.2 |
| Total of correlated substances | | 2.2 |

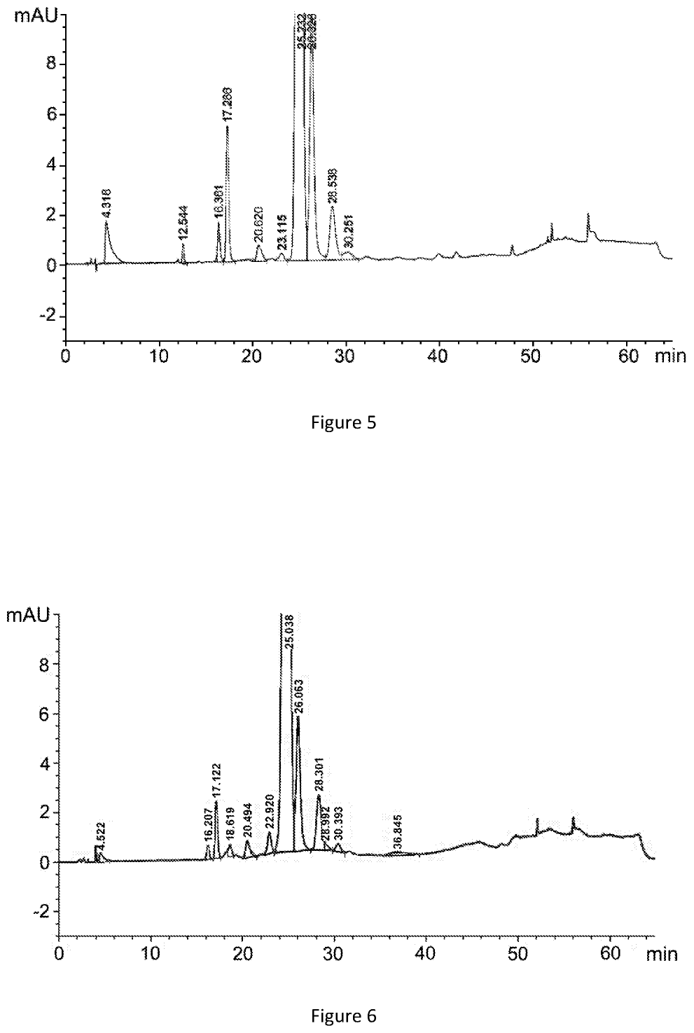

FIG. 5 and FIG. 6 illustrate HPLC chromatograms (tracing the UV signal read at 280 nm) obtained by analyzing, respectively, commercial dalbavancin (Xydalba, Durata Therapeutics) and dalbavancin obtained by applying the method described in this document. Both samples were injected at the same concentration. The chromatogram shown in FIG. 6 (dalbavancin obtained by following the method described in the present description) shows the significant reduction of all the related impurities compared to the chromatogram shown in FIG. 5 (commercial dalbavancin).

Of course, without prejudice to the principle of the invention, the details of construction and the embodiments may be widely varied, without thereby departing from the scope of the invention as defined by the claims that follow.

1. Method for purifying at least one lipoglycopeptide antibiotic comprising the steps of:
i) dissolving said at least one lipoglycopeptide antibiotic in an aqueous solution to form a mixture,
ii) loading said mixture into a chromatographic column comprising a stationary phase, wherein said stationary phase comprises silica functionalized with organic pendants,
iii) eluting the mixture loaded in step ii) using an eluent composition comprising a water-soluble organic solvent obtaining eluate fractions,
iv) selecting the eluate fractions containing the at least one purified lipoglycopeptide antibiotic.

2. Method according to claim 1, wherein the at least one lipoglycopeptide antibiotic is selected in the group consisting of A40926, dalbavancin, teicoplanin, mideplanine (MDL-62873), and ramoplanin.

3. Method according to claim 1, wherein said silica functionalized with organic pendants is selected in the group consisting of octadecyl silyl derivatized silica, octyl silyl derivatized silica, exylphenyl silyl derivatized silica, and butyl silyl derivatized silica, preferably octadecyl silyl derivatized silica.

4. Method according to claim 1, wherein said silica functionalized with organic pendants has a particle size lower than 50 μm, preferably lower than 20 μm.

5. Method according to claim 1, wherein the water-soluble organic solvent contained in the eluent composition of step iii) is selected in the group consisting of methanol, propanol, isopropanol, acetonitrile, acetone.

6. Method according to claim 1, wherein the eluent composition further comprises at least one additional compound selected in the group consisting of ammonium formate, ammonium acetate, triethylamine, formic acid, acetic acid, trifluoroacetic acid, heptafluorobutyric acid, methanesulfonic acid.

7. Method according to claim 1, wherein the eluent composition comprises said water-soluble organic solvent, preferably acetonitrile, in a concentration increasing over time, at least in one interval of conduction time of the elution step iii) or along the entire elution step iii).

8. Method according to claim 1, wherein the elution step iii) is carried out at a pH between 5.0 and 7.0, preferably between 5.2 and 6.5.

9. Method according to claim 1, wherein said stationary phase is subjected, before said step ii), to conditioning with a mobile phase which comprises at least one compound selected in the group consisting of ammonium formate, ammonium acetate or triethylamine salts with formic acid, acetic acid, trifluoroacetic acid.

10. Method according to claim 9, wherein said mobile phase for the conditioning of the stationary phase has a pH between 5.0 and 7.0.

11. Method according to claim 1, wherein the method further comprises the steps of:
v) concentrating the eluate fractions collected in step iv) to obtain a concentrate,
vi) optionally dialyzing the concentrate obtained in step v),
vii) precipitating said concentrate, optionally dialyzed in step vi), with a precipitation solution comprising at least one organic solvent,
viii) drying the precipitate obtained in step vii).

12. Method according to claim 11, wherein said concentration step v) is carried out by means of a membrane nanofiltration system with a molecular cut-off ranging from 100 Da to 1500 Da.

13. Method according to claim 11, wherein said precipitation solution comprises at least one organic solvent selected in the group consisting of acetonitrile, acetone, isopropanol, propanol, and ethanol, preferably acetone.

14. Method according to claim 11, wherein the drying step is carried out by heating at a temperature ranging from 15° C. to 30° C. and, preferably, at a pressure lower than 150 mBar.
